(19) United States
(12) Patent Application Publication
(10) Pub. No.: US 2014/0030342 A1
(43) Pub. Date: Jan. 30, 2014

(54) PROCESS FOR PRODUCING AN IMMUNOGENIC COMPOSITION CONTAINING TETANUS TOXOID

(71) Applicants: Koen De-Heyder, Rixensart (BE); Olivier Staudt, Rixensart (BE)

(72) Inventors: Koen De-Heyder, Rixensart (BE); Olivier Staudt, Rixensart (BE)

(73) Assignee: GLAXOSMITHKLINE BIOLOGICALS SA, Rixensart (BE)

(21) Appl. No.: 14/110,283

(22) PCT Filed: Apr. 5, 2012
(86) PCT No.: PCT/EP12/56377

§ 371 Date: Oct. 7, 2013

(30) Foreign Application Priority Data
Apr. 8, 2011 (GB) 1105981.3

(51) Int. Cl. A61K 9/14; A61K 39/05; A61K 39/295; A61K 39/02; A61K 39/145; A61K 39/29; A61K 39/08; A61K 39/13

(52) U.S. Cl. CPC A61K 9/14; A61K 39/08; A61K 39/05; A61K 39/13; A61K 39/099; A61K 39/145; A61K 39/292; A61K 39/295
USPC 424/489; 424/239.1; 424/238.1; 424/201.1; 424/203.1

(57) ABSTRACT

The present invention relates to the field of vaccines for protecting against tetanus, and in particular processes for the production of vaccines comprising tetanus toxoid adsorbed onto aluminium salts. Processes are provided whereby tetanus toxoid is absorbed onto aluminium salt adjuvant having defined characteristics for optimal results.

### FIELD OF THE INVENTION

The present invention relates to the field of vaccines for protecting against tetanus, and in particular processes for the production of vaccines comprising tetanus toxoid adsorbed onto aluminium salts.

### BACKGROUND

Vaccines are known which can prevent Clostridium tetani typically in combination with Bordetella pertussis, Corynebacterium diphtheriae. Such vaccines may comprise one or more antigens derived from Clostridium tetani, Bordetella pertussis and/or Corynebacterium diphtheriae adsorbed onto aluminium salts. The present inventors have surprisingly found that the protein adsorption and/or crystal size as measured by X-ray diffraction of aluminium salts is important in the immunogenicity of antigens adsorbed to said aluminium salts and in particular toxoids derived from Clostridium tetani ([e.g.] tetanus toxoid).

### SUMMARY OF INVENTION

Accordingly, the present invention provides a process for producing an immunogenic composition comprising tetanus toxoid comprising the step of adsorbing the tetanus toxoid onto an aluminium salt particle wherein the aluminium salt particle has a protein adsorption capacity between 2.5 and 3.5 mg protein/mg aluminium salt.

In a further aspect of the invention, there is provided a process for producing an immunogenic composition comprising tetanus toxoid comprising the step of adsorbing the tetanus toxoid onto an aluminium salt particle wherein the aluminium salt particle has a crystal size of between 2.8 and 5.7 nm as measured by X-ray diffraction.

In a further aspect, the invention provides a process for sterilising an aluminium salt adjuvant which comprises a step of irradiation and comprises no steps of autoclaving.

### BRIEF DESCRIPTION OF THE DRAWINGS

FIG. 1 Zeta potential pH 7.0 (mV) vs protein adsorption (mg BSA/mg Al³⁺).

FIG. 2 Effect of radiation on protein adsorption and surface charge (ZP).

### DETAILED DESCRIPTION OF THE INVENTION

The present inventors have found that the protein adsorption, zeta potential and/or crystal size of aluminium salts on which tetanus toxoid is adsorbed are very important for the immunological characteristics of the tetanus toxoid vaccine. Accordingly, the present invention provides a process for producing an immunogenic composition comprising tetanus toxoid, comprising the step of adsorbing the tetanus toxoid onto an aluminium salt particle wherein the aluminium salt particle has a protein adsorption capacity between 2.5 and 3.5 mg protein/mg aluminium salt.

Tetanus toxoids (TT) and their methods of preparation are well known in the art. In one embodiment TT is produced by purification of the toxin from a culture of Clostridium tetani followed by chemical detoxification, but it is alternatively made by purification of a recombinant, or genetically detoxified, analogue of the toxin (for example, as described in EP 209281). A preferred method of detoxification is as follows. Following fermentation, the broth is filtered on a 0.1-0.3 μm filter in the presence of Diatomite as filter aid. The harvest is clarified through a 0.22 μm filter, concentrated and diafiltered on 30 kD flatsheet membranes against 10 volumes of phosphate buffer (20 mM-pH 7.3). The diafiltered toxin is then detoxified for 4 weeks at 37° C. in the following conditions: formaldehyde 20 mM-lysine 3 mM-potassium phosphate 100 mM-initial pH 7.3-500 Lf/ml. The resulting toxoid is purified by ammonium sulfate fractionation, concentrated and diafiltered (30 kD) against WFI to remove ammonium sulfate. NaCl is added to a final concentration of 0.9%, the pH is adjusted to 7.3 and the purified tetanus toxoid is sterile filtered.

Any suitable tetanus toxoid may be used. ‘Tetanus toxoid’ may encompass immunogenic fragments of the full-length protein (for instance Fragment C—see EP 478602).

The tetanus toxoid of the invention is adsorbed onto an aluminium salt. In a particular embodiment of the invention the aluminium salt is aluminium hydroxide. In another embodiment, the tetanus toxoid of the invention may be adsorbed onto an aluminium salt such as aluminium phosphate. In a further embodiment the tetanus toxoid may be adsorbed onto a mixture of both aluminium hydroxide and aluminium phosphate.

Methods of adsorbing protein including tetanus toxoids onto aluminium salts are well known to the skilled person (for example vaccine preparation is generally described in Vaccine Design “The subunit and adjuvant approach” (eds Powell M. F. & Newman M. J.) (1995) Plenum Press New York).

In a particular embodiment of the invention the aluminium salt has a protein adsorption capacity between 2.5 and 3.5, 2.6 and 3.4, 2.7 and 3.3 or 2.9 and 3.2, for example a protein adsorption capacity of 2.5, 2.6, 2.7, 2.8, 2.9, 3.0, 3.1, 3.2, 3.3, 3.4 or 3.5 mg protein/mg aluminium salt. The aluminium salt may have a protein adsorption capacity between 2.5 and 3.7, 2.6 and 3.6, 2.7 and 3.5 or 2.8 and 3.4, for example a protein adsorption capacity of 3.6 mg protein (BSA)/mg aluminium salt. Protein adsorption capacity of the aluminium salt can be measured by any means known to the skilled person. In a particular embodiment of the invention, the protein adsorption capacity of the aluminium salt is measured using the method as described in Example 1 (which utilises BSA) or variations thereof. In a particular embodiment of the invention, the protein adsorption capacity of the aluminium salt is between 2.9 and 3.2 mg BSA/mg aluminium salt.

In a further embodiment of the invention, the aluminium salt of the invention has a crystal size of between 2.8 and 5.7 nm as measured by X-ray diffraction, for example 2.9 to 5.6 nm, 2.8 to 3.5 nm, 2.9 to 3.4 nm or 3.4 to 5.6 nm. X-ray diffraction is well known to the skilled person. In a particular embodiment of the invention the crystal size is measured using the method described in Example 1.

In a further embodiment of the invention, the aluminium salt has a Zeta potential at pH 7 of between about 17 and 23 mV, 18 and 22 mV or 19 and 21 mV, for example of 17, 18, 19, 20, 21, 22, or 23 mV. The aluminium salt may have a Zeta potential at pH 7 of between 14 and 22 mV, 15 and 21 mV or 16 and 20 mV, for example of 14, 15 or 16mV. The Zeta potential can be measured by any means known to the skilled person, for example by Digital Light Scattering (DLS). In a particular embodiment of the invention the Zeta potential is measured using the method as described in Example 1.

Aluminium salts having at least a protein adsorption capacity within the above ranges, and optionally with a crystal size and/or a zeta potential within the above ranges, have been found to be optimal for the formulation of combination vaccines containing tetanus toxoid, in terms of tetanus toxoid potency as well as the immunogenicity of co-formulated antigens. Increased potency of antigens such as tetanus toxoid opens up the possibility to use lower amounts of antigen to achieve the same level of immune response, facilitating antigen-sparing.

Aluminium salts of the invention can be made by any method known to the skilled person (see for example vaccine preparation is generally described in Vaccine Design “The subunit and adjuvant approach” (eds Powell M. F. & Newman M. J.) (1995) Plenum Press New York, U.S. Pat. No. 4,882,140A & U.S. Pat. No. 4,826,606A). The skilled person knows how to alter the protein adsorption, crystal size and/or surface charge (Zeta potential at pH 7) and therefore how to make aluminium salts exhibiting these characteristics within defined parameters.

Alternatively aluminium salts for use in the processes of the invention can be commercially sourced, for example Rehydragel™ HS (3% aluminium hydroxide in water [General Chemical]) or Alhydrogel™ 85 (Brenntag BioSector [Denmark]).

In a particular embodiment of the invention, the aluminium salts as described herein (for example Rehydragel™ HS or Alhydrogel™ 85) have not been autoclaved following purchase. (Alhydrogel™ 85 is autoclaved by the manufacturer (i.e. prior to purchase), whereas Rehydragel™ HS is instead sterilised by irradiation by the manufacturer.) In order to produce a sterile aluminium salt of the invention, the aluminium salt is sterilised by other means, in particular by irradiation. The aluminium salt may be irradiated to give a sterile aluminium salt using ultra violet light (UV), gamma (λ) radiation from a radioisotope source (e.g. cobalt-60), beta (β) radiation, an electron-beam or X-ray irradiation. In a particular embodiment, the aluminium salts of the invention are sterilised by gamma radiation. In another embodiment, there is provided a process for sterilising an aluminium salt adjuvant which comprises autoclaving said adjuvant only once. In this embodiment, the adjuvant may be autoclaved for the minimum time necessary to achieve sterilisation, for example for no longer than about 90 minutes from the beginning to the end of the autoclave cycle. Optionally, the once-autoclaved adjuvant may also be sterilised by irradiation.

The present inventors have shown that autoclaving aluminium salts of the invention can reduce the immunogenicity of proteins (in particular tetanus toxoid) adsorbed to said aluminium salt. Accordingly, in one embodiment of the invention there is provided a process for sterilising an aluminium salt adjuvant which comprises a step of irradiation and comprises no steps of autoclaving.

In a further embodiment, the invention provides a process for producing an immunogenic composition comprising tetanus toxoid and diphtheria toxoid, comprising the step of adsorbing the diphtheria toxoid onto an aluminium salt particle wherein the aluminium salt particle is as described herein.

Diphtheria toxoids (DT) and their methods of preparation are well documented. Any suitable diphtheria toxoid may be used. For instance, DT may be produced by purification of the toxin from a culture of Corynebacterium diphtheriae followed by chemical detoxification, but is alternatively made by purification of a recombinant, or genetically detoxified analogue of the toxin (for example, CRM197, or other mutants as described in U.S. Pat. No. 4,709,017, U.S. Pat. No. 5,843,711, U.S. Pat. No. 5,601,827, and U.S. Pat. No. 5,917,017). A preferred method of detoxification is as follows. Following fermentation, the Diphtheria toxin is harvested by TFF 0.45 μm, clarified through a 0.22 μm filter, concentrated and diafiltered on 10 kD flatsheet membranes against 10 volumes of phosphate buffer (20 mM-pH 7.2). The diafiltered toxin is then detoxified for 6 weeks at 37° C. in the following conditions: formaldehyde 50 mM-lysine 25 mM-potassium phosphate 50 mM-initial pH 7.2-300 Lf/ml. The resulting toxoid is purified by ammonium sulfate fractionation, concentrated and diafiltered (30 kD) against WFI to remove ammonium sulfate. NaCl is added to a final concentration of 0.9%, the pH is adjusted to 7.3 and the purified diphtheria toxoid is sterile filtered.

In a particular embodiment there is provided a process of the invention wherein the tetanus and diphtheria toxoids are adsorbed either separately or together onto the aluminium salt. In a further embodiment there is provided a process of the invention wherein the tetanus toxoid of the invention is adsorbed onto an aluminium salt of the invention and wherein the diphtheria toxoid is adsorbed onto a different aluminium salt which may be an aluminium salt of the invention or any other aluminium salt.

In a further embodiment, there is provided a process of the invention further comprising the step of formulating the immunogenic composition with an inactivated polio vaccine. The inactivated polio vaccine (IPV) may comprise IPV type 1 or IPV type 2 or IPV type 3, or IPV types 1 and 2, or IPV types 1 and 3, or IPV types 2 and 3, or IPV types 1, 2 and 3.

Methods of preparing inactivated poliovirus (IPV) are well known in the art. In one embodiment, IPV should comprise types 1, 2 and 3 as is common in the vaccine art, and may be the Salk polio vaccine which is inactivated with formaldehyde (see for example, Sutter et al, 2000, Pediatr. Clin. North Am. 47:287; Zimmerman & Spann 1999, Am Fam Physician 59:113; Salk et al., 1954, Official Monthly Publication of the American Public Health Association 44(5):563; Hennesen, 1981, Develop. Biol. Standard 47:139; Budowsky, 1991, Adv. Virus Res. 39:255). Alternatively, IPV may be made using Sabin strains (Sabin-IPV; Kersten at al (1999), Vaccine 17:2059).

In one embodiment the IPV is not adsorbed (e.g. before mixing with other components). In another embodiment, the IPV component(s) of the invention may be adsorbed onto an aluminium salt such as aluminium hydroxide (e.g. before or after mixing with other components). In another embodiment, the IPV component(s) of the invention may be adsorbed onto an aluminium salt such as aluminium phosphate. In a further embodiment the IPV component(s) may be adsorbed onto a mixture of both aluminium hydroxide and aluminium phosphate. If adsorbed, one or more IPV components may be adsorbed separately or together as a mixture. In a further embodiment, the IPV is adsorbed onto an aluminium salt/particle as described herein.

In a further embodiment, there is provided a process of the invention further comprising the step of formulating the immunogenic composition with pertactin. Pertactin (the 69 kDa antigen of pertussis) is an outer membrane protein which is heat-stable and can be prepared by methods known in the art (see EP0162639). The pertactin is optionally adsorbed onto an aluminium salt particle. In one embodiment of the invention the pertactin is adsorbed to aluminium hydroxide. In a particular embodiment of the invention, the pertactin is adsorbed onto an aluminium salt as described herein.

In a further embodiment, there is provided a process of the invention further comprising the step of formulating the immunogenic composition with filamentous haemagglutinin (FHA). FHA can be prepared in methods well known in the art (see methods disclosed and referenced in WO/1990/013313 (U.S. Pat. No. 74,792,83)). The FHA is optionally adsorbed onto an aluminium salt particle. In one embodiment of the invention the FHA is adsorbed to aluminium hydroxide. In a particular embodiment of the invention, the FHA is adsorbed onto an aluminium salt as described herein.

In a further embodiment, there is provided a process of the invention further comprising the step of formulating the immunogenic composition with pertussis toxoid. Methods of producing pertussis toxoid are well known to the skilled person. Pertussis toxin may be detoxified by a well known method of formaldehyde treatment or by means of mutations (PT derivative). Substitutions of residues within the S1 subunit of the protein have been found to result in a protein which retains the immunological and protective properties of the pertussis toxin, but with reduced or no toxicity (EP 322533). The detoxifying mutations discussed in the claims of EP322533 are examples of the PT detoxified mutants of the present invention. The pertussis toxoid is optionally adsorbed onto an aluminium salt particle. In one embodiment of the invention the pertussis toxoid is adsorbed to aluminium hydroxide. In a particular embodiment of the invention, the pertussis toxoid is adsorbed onto an aluminium salt as described herein.

In a further embodiment, there is provided a process of the invention further comprising the step of formulating the immunogenic composition with a capsular saccharide from Haemophilus influenzae type b (Hib), optionally adsorbed onto an aluminium salt particle.

The polyribosyl ribitol phosphate capsular saccharide (PRP) from Haemophilus influenzae type b may be conjugated to a carrier protein. The saccharide is a polymer of ribose, ribitol and phosphate. The Hib antigen may optionally be adsorbed onto aluminium phosphate as described in WO97/00697, or may be unadsorbed as described in WO02/00249 or may not have undergone a specific process of adsorption.

By an antigen being ‘unadsorbed onto an aluminium adjuvant salt’ (“unadsorbed” or “not adsorbed”) herein it is meant for example that an express or dedicated adsorption step for the antigen on fresh aluminium adjuvant salt is not involved in the process of formulating the composition.

Hib may be conjugated to any carrier which can provide at least one T-helper epitope, and may be tetanus toxoid, diphtheria toxoid, CRM-197 (diphtheria toxin mutant) or Protein D from non-typeable H. influenzae (EP0594610).

In a further embodiment, there is provided a process of the invention further comprising the step of formulating the immunogenic composition with a hepatitis B surface antigen.

The preparation of Hepatitis B surface antigen (HBsAg) is well documented. See for example, Hartford et al, 1983, Develop. Biol. Standard 54:125; Gregg et al., 1987, Biotechnology 5:479; EP0226846; EP0299108. It may be prepared as follows. One method involves purifying the antigen in particulate form from the plasma of chronic hepatitis B carriers, as large quantities of HBsAg are synthesised in the liver and released into the blood stream during an HBV infection. Another method involves expressing the protein by recombinant DNA methods. The HBsAg may be prepared by expression in the Saccharomyces cerevisiae yeast, pichia, insect cells (e.g. Hi5) or mammalian cells. The HBsAg may be inserted into a plasmid, and its expression from the plasmid may be controlled by a promoter such as the “GAPDH” promoter (from the glyceraldehyde-3-phosphate dehydrogenase gene). The yeast may be cultured in a synthetic medium. HBsAg can then be purified by a process involving steps such as precipitation, ion exchange chromatography, and ultrafiltration. After purification, HBsAg may be subjected to dialysis (e.g. with cysteine). The HBsAg may be used in a particulate form.

As used herein the expression “Hepatitis B surface antigen” or “HBsAg” includes any HBsAg antigen or fragment thereof displaying the antigenicity of HBV surface antigen. It will be understood that in addition to the 226 amino acid sequence of the HBsAg S antigen (see Tiollais et al, 1985, Nature 317:489 and references therein) HBsAg as herein described may, if desired, contain all or part of a pre-S sequence as described in the above references and in EP0278940. In particular, the HBsAg may comprise a polypeptide comprising an amino acid sequence comprising residues 133-145 followed by residues 175-400 of the L-protein of HBsAg relative to the open reading frame on a Hepatitis B virus of ad serotype (this polypeptide is referred to as L*; see EP0414374). HBsAg within the scope of the invention may also include the preS1-preS2-S polypeptide described in EP0198474 (Endotronics) or analogues thereof such as those described in EP0304578 (McCormick and Jones). HBsAg as used herein can also refer to mutants, for example the “escape mutant” described in WO 91/14703 or EP0511855A1, especially HBsAg wherein the amino acid substitution at position 145 is to arginine from glycine.

The HBsAg may be in particle form. The particles may comprise for example S protein alone or may be composite particles, for example L*, S) where L* is as defined above and S denotes the S-protein of HBsAg. The said particle is advantageously in the form in which it is expressed in yeast.

In one embodiment, HBsAg is the antigen used in EngerixB™ (GlaxoSmithKline Biologicals S.A.), which is further described in WO93/24148.

Hepatitis B surface antigen may optionally be adsorbed onto an aluminium salt, in particular aluminium phosphate, which may be done before mixing with the other components (described in WO93/24148). The Hepatitis B component should be substantially thiomersal free (method of preparation of HBsAg without thiomersal has been previously published in EP1307473).

In a further embodiment of the invention, there is provided a process for sterilising an aluminium salt adjuvant which comprises a step of irradiation and comprises no steps of autoclaving. Sterilisation by irradiation may be performed by any method known to the skilled person and in particular by any of the irradiation methods described herein.

In a further embodiment of the invention, there is provided such a process wherein the aluminium salt is aluminium hydroxide, wherein size of unit crystal is between 2.8 and 5.7 nm as measured by X-ray diffraction, for example 2.9 to 5.6 nm, 2.8 to 3.5nm, 2.7 to 3.4 nm or 3.4 to 5.6 nm and wherein irradiation is selected from the group of ultra violet light (UV), gamma (A) radiation from a radioisotope source (e.g. cobalt-60), beta (β) radiation, an electron-beam or X-ray irradiation.

In a further embodiment of the invention, there is provided a process for making a vaccine comprising one or more of diphtheria toxoid, tetanus toxoid, pertussis toxoid, filamentous haemagglutinin and pertactin, wherein one or more of diphtheria toxoid, tetanus toxoid, pertussis toxoid, filamentous haemagglutinin and pertactin are adsorbed onto an aluminium adjuvant made by any of the processes for sterilising an aluminium salt adjuvant which comprise a step of irradiation and comprise no steps of autoclaving described herein.

In a particular embodiment of the invention, there is provided a process as described herein wherein tetanus toxoid and/or diphtheria toxoid are present and are adsorbed to an aluminium adjuvant made by any of the processes for sterilising an aluminium salt adjuvant which comprise a step of irradiation and comprise no steps of autoclaving described herein.

In a further embodiment of the invention, there is provided a process as described herein wherein diphtheria toxoid, tetanus toxoid, pertussis toxoid, filamentous haemagglutinin and pertactin are all present and are adsorbed to an aluminium adjuvant made by any of the processes for sterilising an aluminium salt adjuvant which comprise a step of irradiation and comprise no steps of autoclaving described herein.

The terms “comprising”, “comprise” and “comprises” herein is intended by the inventors to be substitutable with the terms “consisting of”, “consist of and “consists of”, respectively, in every instance.

The term “immunogenic composition” is optionally substitutable with the term “vaccine” and vice versa.

The following examples illustrate, but do not the limit, the scope of the invention.

### EXAMPLES

Note in relation to the following Examples: as discussed above, Alhydrogel™ 85 (and Alhydrogel™) is autoclaved prior to purchase, whilst ReHydragel™ HS and the other “ReHydragel™” characterised aluminium salts are not. Hence, where an “Alhydrogel™” product is indicated to have been autoclaved or re-autoclaved, this means autoclaved after purchase (i.e. for a second time). Where a “ReHydragel™” product is stated to have been autoclaved, this means a first autoclaving carried out post-purchase.

### Example 1

### Characterisation of Aluminium Salts

The protein adsorption capacity, surface charge (zeta potential; ZP) and crystal sizes were determined for a variety of aluminium hydroxides.

1.1 BSA Adsorption Capacity of Aluminium Hydroxide

Stock solutions of bovine serum albumin (BSA), comprising 6 mg/ml in MilliQ water and Aluminium hydroxide 1 mgAl³⁺/ml in MilliQ water, were prepared.

The aluminium content was determined by atomic spectrophotometry with nitrous flame. A range of standard solutions of aluminium prepared in Hydrochloric acid (HCl) was used to establish the standard curve. HCl was used as blank. A sample of known amount of aluminium was used as positive control. The test samples and positive control were diluted in HCl to obtain a final concentration of aluminium within the range of the standard curve. After mineralization by heating, samples were cooled to room temperature and brought to volume with purified water. The aluminium concentration was then determined using a nitrous oxide-acetylene flame. The aluminium content in the test solution was determined from the standard curve.

Samples were prepared with different ratios of BSA/aluminium, for example:

| | | Sample | Sample | Sample | Sample | Sample |
|---|---|---|---|---|---|---|
| | Blank | 1 | 2 | 3 | 4 | 5 |
| Ratio μg BSA/ | 0 | 2.4 | 2.7 | 3 | 3.3 | 3.6 |
| μgAl3+ | | | | | | |
| Vol water (ml) | 4.54 | 2.54 | 2.29 | 2.04 | 1.79 | 1.54 |
| Vol AlOOH stock | 0.46 | 0.46 | 0.46 | 0.46 | 0.46 | 0.46 |
| (ml) | | | | | | |
| Vol BSA Stock | 0.00 | 2.00 | 2.25 | 2.50 | 2.75 | 3.00 |
| (ml) | | | | | | |
| V Total | 5.00 | 5.00 | 5.00 | 5.00 | 5.00 | 5.00 |

(In the above table “Vol” and “V” in the left column mean “volume”.)

The ratio of BSA/Al³⁺ was adapted depending on the assumed adsorption capacity of the aluminium salt:

| | Blc | S1 | S2 | S3 | S4 | S5 |
|---|---|---|---|---|---|---|
| Rehydragel HS | 0.0 | 2.6 | 3.0 | 3.4 | 3.8 | 4.2 |
| Alhydrogel 85 | 0.0 | 2.4 | 2.7 | 3.0 | 3.3 | 3.6 |
| Rehydragel PM | 0.0 | 2.0 | 2.5 | 3.0 | 3.5 | 4.0 |
| Rehydragel AB | 0.0 | 2.5 | 2.9 | 3.3 | 3.7 | 4.1 |
| Rehydragel HPA | 0.0 | 3.0 | 3.6 | 4.2 | 4.8 | 5.4 |
| Rehydragel LV | 0.0 | 3.5 | 3.9 | 4.3 | 4.7 | 5.1 |
| Alhydrogel | 0.0 | 1.8 | 2.1 | 2.4 | 2.7 | 3.0 |

The pH was adjusted to 6.1+/−0.1 with NaOH 0.05N or HCl 0.1N. The BSA was allowed to adsorb to the aluminium hydroxide overnight (16+/−4 h) at room temperature. The samples were then centrifuged at 4000 rpm for 20 mins until a clear supernatant was observed. The BSA concentration in the supernatant was measured by BCA (bicinchoninic acid) protein assay (Pierce) and the amount of BSA adsorbed on aluminium hydroxide was calculated by calculating the difference between the initial concentration and the concentration in the supernatant. The BSA adsorbed was plotted as a function of BSA in the supernatant to provide a curve showing a plateau. The height of the plateau was calculated as the adsorption capacity.

1.2 Surface Charge (ZP) Measurement of Aluminium Salts

Zeta potential (ZP) was measured using a DLS Malvern Zetasizer Nano S. All samples and diluents were brought to room temperature. Aluminium hydroxide was diluted in MilliQ water to a concentration of 0.05 to 0.2 mg Al³⁺/ml. The equipment was calibrated (−68 mV standard) and the ZP was measured using the following measurement parameters:
Measurement type: Zeta Potential
Cell: DTS1060
Sample: Model Smoluchowsky, Al(OH)3
Dispersant: Water
T°: 21° C. (room temperature)
Equilibration duration: Automatic
Number of measurements: 3 or 5
Delay between measurements: 30-60 secs
Voltage: Automatic
Result calculation: Monomodal

1.3 Measurement of the Crystal Size by X-ray Detraction

Samples were prepared by removing any NaCl (by dialysis) and drying (either by desiccation or lyophilisation). The crystal size was then measured using the following standard X-ray analysis parameters:
Angle 5 to 90°, step size=0.02°, step scan=2,4 s, continuous mode
170 min data acquisition
6 mm diameter support
Tube power, 40V 40 mA

The crystal size was then calculated as follows:

Sherrer's law relates width at half height of diffraction peaks to the average crystallite size:

D=(k*λ)/(β*cos(θ))

where

D: average crystal size

k: form (shape) factor, usually=1

λ: wavelength 0,154056 nm (Kalpha Cu)

β: corrected peak width radian

θ: Bragg angle (here)19,168°)

1.4 Results

The adsorption capacity, surface charge and crystal size of different aluminium hydroxides are shown in the two tables below. The values shown are averages from multiple measurements encompassing at least one (often multiple) batch(es) of the respective aluminium hydroxides. The second table is analogous to the first but was produced later after further measurements had been performed.

| | Adsorption | | |
|---|---|---|---|
| | Capacity | Zeta Potential | Crystal |
| | mg BSA/mg Al | pH 7.0 mV | size nm |
| Alhydrogel ™ | 2.2 | 20 | 5.8 |
| (Not re-autoclaved) | | | |
| Alhydrogel ™ | 2.1 | 19 | 5.3 |
| (Re-autoclaved) | | | |
| Alhydrogel ™ 85 | 3.0 | 19 | 5.6 |
| (Not re-autoclaved) | | | |
| Alhydrogel ™ 85 | 2.7 | 10 | 4.9 |
| (Re-autoclaved) | | | |
| Rehydragel ™ HPA | 5.3 | 30 | 1.9 |
| Rehydragel ™ LV | 4.1 | 30 | 3.4 |
| Rehydragel ™ PM | 2.8 | 12 | 3.0 |
| Rehydragel ™ AB | 3.1 | 23 | 3.2 |
| Rehydragel ™ HS | 3.5 | 19 | 2.9 |

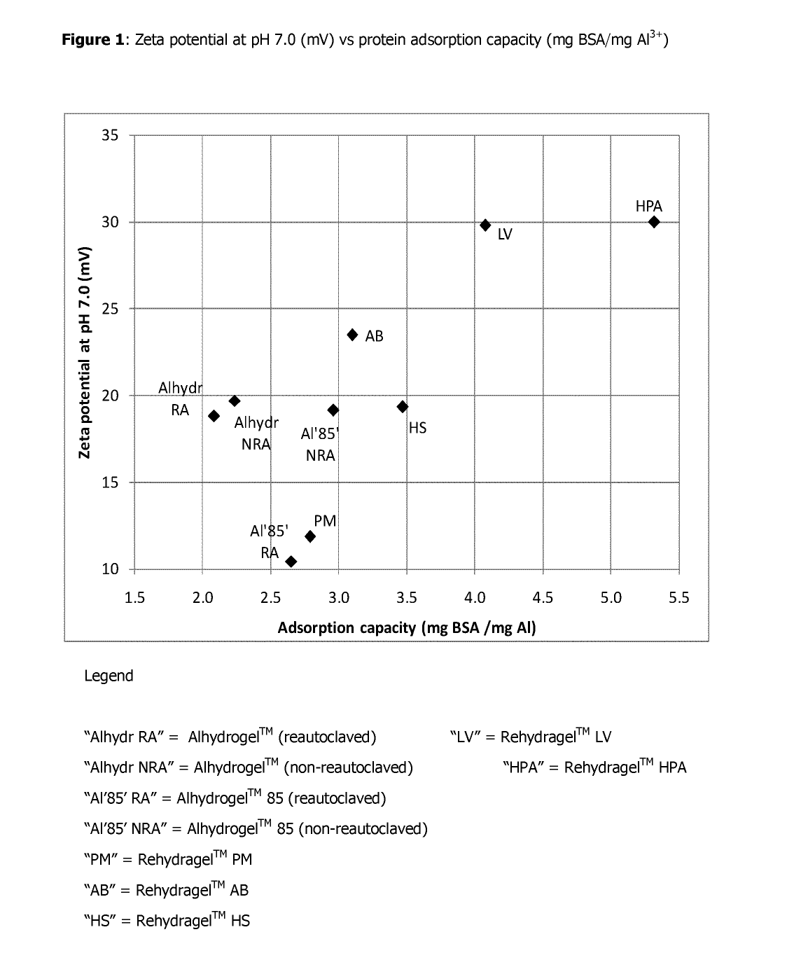

FIG. 1 shows Zeta potential plotted against protein adsorption capacity using the values in the above table.

| | Adsorption | | |
|---|---|---|---|
| | Capacity | Zeta Potential | Crystal |
| | mg BSA/mg Al | pH 7.0 mV | size nm |
| Alhydrogel ™ | 2.6 | 18 | 5.8 |
| (Not re-autoclaved) | | | |
| Alhydrogel ™ | 2.1 | 16 | 5.3 |
| (Re-autoclaved) | | | |
| Alhydrogel ™ 85 | 3.1 | 16 | 5.6 |
| (Not re-autoclaved) | | | |
| Alhydrogel ™ 85 | 2.7 | 15 | 4.9 |
| (Re-autoclaved) | | | |
| Rehydragel ™ HPA | 5.3 | 32 | 1.9 |
| Rehydragel ™ LV | 4.1 | 32 | 3.4 |
| Rehydragel ™ PM | 2.8 | 12 | 3.0 |
| Rehydragel ™ AB | 3.1 | 27 | 3.2 |
| Rehydragel ™ HS | 3.6 | 20 | 2.9 |

1.5 Effects of Autoclaving

The effects of autoclaving aluminium salts were determined for the different lots of Rehydragel™ HS and Alhydrogel™ 85. As shown in the table below, autoclaving reduced the adsorption capacity of the aluminium salts.

Adsorption capacity (mg BSA/mg Al³⁺)

| Description | Non autoclaved | Autoclaved |
|---|---|---|
| Rehydragel HS Lot 1 | 3.0 | 2.7 |
| Rehydragel HS Lot 2 | 3.1 | 2.8 |
| Rehydragel HS Lot 3 | 3.4 | 2.6 |
| Alhydrogel 85 Lot 1 | 2.9 | 2.4 |
| Alhydrogel 85 Lot 2 | 3.2 | 2.8 |
| Alhydrogel 85 Lot 3 | 2.6 | 2.5 |

As shown in the table below, surface charge measured as Zeta Potential was also impacted by autoclaving.

Zeta Potential (mV) pH 7.0

| Description | Non autoclaved | Autoclaved |
|---|---|---|
| Rehydragel HS Lot 1 | 21 | 17 |
| Rehydragel HS Lot 2 | 19 | 18 |
| Rehydragel HS Lot 3 | 20 | 16 |
| Alhydrogel 85 Lot 1 | 16 | 16 |
| Alhydrogel 85 Lot 2 | 20 | — |
| Alhydrogel 85 Lot 3 | 17 | — |
| Alhydrogel 85 Lot 4 | 15 | 14 |

1.6 Effects of Irradiation

As autoclaving affected the surface charge and protein adsorption of the aluminium salts the effects of sterilisation by radiation were investigated.

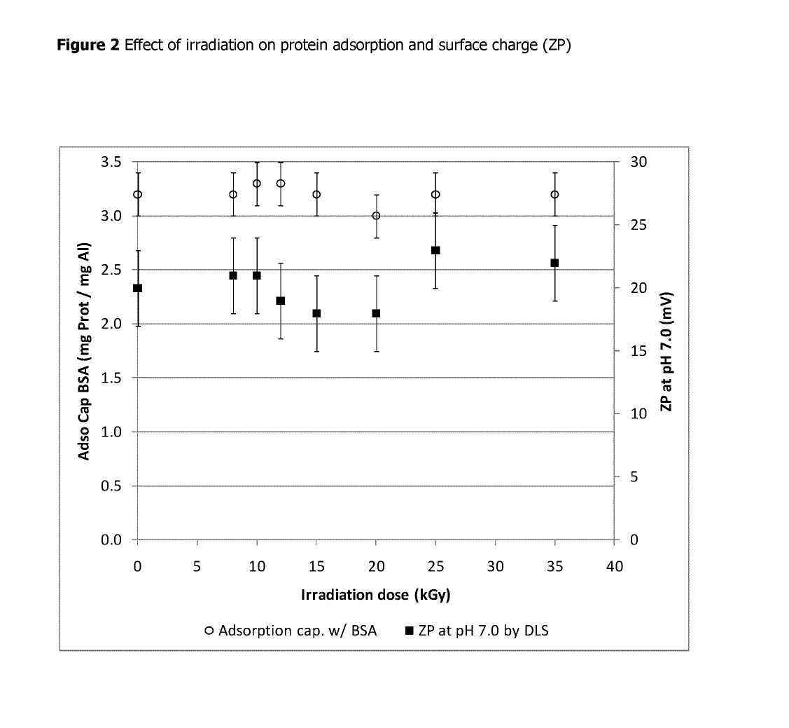

FIG. 2 shows that irradiation affects neither adsorption capacity, nor surface charge.

### Example 2

### Potency of TT

TT Potency (potency of tetanus toxoid) of different aluminium salts in Diphtheria, Tetanus, acellular Pertussis & Inactivated Polio Virus (DTaP IPV) combination vaccine.

A mixture of DT and TT were adsorbed onto different aluminium salts and the TT potency was tested. TT potency was tested according to the test as required by the European Pharmacopoeia (method number 2.07.08). The results are presented in the table below. (The Rehydragel™ HS DT concentrate in the bottom row was stored at 4° C. whilst awaiting formulation into DTaP IPV, while all other DT concentrates were matured for 7 days at 37° C. before final formulation. In this table, as in the other tables in Example 2, “DT” means “diphtheria toxoid and tetanus toxoid”; “Potency T” means “potency of tetanus toxoid”; “RP” means “relative potency”; and “LCL” and “HCL” respectively mean “low-” and “high-confidence limit”.)

| | Adsorption | | | |
|---|---|---|---|---|
| Aluminium hydroxide | capacity | Potency T (IU/dose) | | |
| for DT Adsorption | (mg BSA/mg Al) | LCL | RP | HCL |
| Rehydragel ™ HS | 4.0 | 70 | 94 | 126 |
| Alhydrogel ™ ‘85’ | 3.1 | 71 | 103 | 148 |
| Alhydrogel ™ ‘85’ | (2.7) | 57 | 78 | 108 |
| autoclaved | | | | |
| Alhydrogel ™ | (2.1) | 43 | 62 | 86 |
| autoclaved | | | | |
| Rehydragel ™ HS | 4.0 | 82 | 115 | 163 |

(Note: the adsorption capacity values given in brackets in the above table were not available and were estimated based on experience available on the corresponding AlOOH.)

In a second experiment, two different T toxoids were used. (The Alhydrogel™ DT concentrate autoclaved in the top row for TT1 was stored at 4° C. whilst awaiting formulation into DTaP IPV, while all other DT concentrates were matured for 7 days at 37° C. before final formulation.)

| | Adsorption | | | |
|---|---|---|---|---|
| Aluminium hydroxide | capacity | Potency T (IU/dose) | | |
| for DT Adsorption | (mg BSA/mg Al) | LCL | RP | HCL |

Tetanus toxoid 1

| | | | | |
|---|---|---|---|---|
| Alhydrogel ™ | (2.1) | 50 | 74 | 113 |
| autoclaved | | | | |
| Alhydrogel ™ 85 | 2.4 | 59 | 84 | 123 |
| autoclaved | | | | |
| Rehydragel ™ HS | 3.1 | 87 | 130 | 199 |

Tetanus toxoid 2

| | | | | |
|---|---|---|---|---|
| Alhydrogel ™ 85 | 2.8 | 52 | 75 | 110 |
| autoclaved | | | | |
| Rehydragel ™ HS | 3.4 | 81 | 125 | 196 |

(Note: the adsorption capacity values given in brackets in the above table were not available and were estimated based on experience available on the corresponding AlOOH.)

In a third experiment, two sets of DT and TT, wherein the antigens were produced using different detoxification methods, were used. (The DT concentrates were stored at 37° C. for 7 days before final formulation.)

| | Adsorption | | | |
|---|---|---|---|---|
| Aluminium hydroxide | capacity | Potency T (IU/dose) | | |
| for DT Adsorption | (mg BSA/mg Al) | LCL | RP | HCL |

DT and TT type 1

| | | | | |
|---|---|---|---|---|
| Alhydrogel ™ | (2.1) | 95 | 145 | 216 |
| autoclaved | | | | |
| Rehydragel ™ HS | 3.4 | 99 | 151 | 225 |
| Alhydrogel ™ ‘85’ | 3.5 | 103 | 153 | 223 |
| irradiated | | | | |

DT and TT type 2

| | | | | |
|---|---|---|---|---|
| Alhydrogel ™ | (2.1) | 49 | 75 | 112 |
| autoclaved | | | | |
| Rehydragel ™ HS | 3.4 | 56 | 82 | 120 |
| Alhydrogel ™ ‘85’ | 3.5 | 56 | 82 | 120 |
| irradiated | | | | |

TT Potency of different aluminium salts in DTaP HBV (Hepatitis B virus) IPV combination vaccine.

Study 1

DT and TT were adsorbed onto different aluminium salts and the TT potency was tested. TT potency was tested according to the test as required by the European Pharmacopoeia (method number 2.07.08). The results are presented in the table below. (All of the DT concentrates were kept at room temperature for 3-4 days, and then at 2-8° C. awaiting formulation into DTaP HBV IPV. The upper and lower potency values for each of Alhydrogel™ 85 autoclaved and Rehydragel™ HS respectively represent DT concentrates using two different D toxoids.)

| | Adsorption | | | |
|---|---|---|---|---|
| Aluminium hydroxide | capacity | Potency T (IU/dose) | | |
| for DT Adsorption | (mg BSA/mg Al) | LCL | RP | HCL |
| Alhydrogel ™ | (2.1) | 74 | 110 | 154 |
| Autoclaved | | | | |
| Alhydrogel ™ ‘85’ | 2.8 | 77 | 117 | 166 |
| Autoclaved | | | | |
| | | 92 | 132 | 185 |
| Rehydragel ™ HS | 3.4 | 114 | 159 | 220 |
| | | 118 | 170 | 242 |
| Rehydragel ™ HS | 2.6 | 123 | 168 | 230 |
| Autoclaved | | | | |

(Note: the adsorption capacity values given in brackets in the above table were not available and were estimated based on experience available on the corresponding AlOOH.)

Study 2

DT and TT were adsorbed onto different aluminium salts and the TT potency was tested. TT potency was tested according to the test as required by the European Pharmacopoeia (method number 2.07.08). The results are presented in the table below. (All of the DT concentrates were matured for 7 days at 37° C. before final formulation into DTaP HBV IPV. Four different TTs were used (toxoided in different ways: Tetanus TTS03-FA02, Tetanus TTS03-FA4, Tetanus TTS03-FA3 and Tetanus TTS02-FA21). The bottom row (Rehydragel™ HS) for the Tetanus TTS03-FA02 toxoid used a reduced amount of DT relative to the other samples.)

| | Adsorption | | | |
|---|---|---|---|---|
| Aluminium hydroxide | capacity | Potency T (IU/dose) | | |
| for DT Adsorption | (mg BSA/mg Al) | LCL | RP | HCL |

Tetanus TTS03-FA02

| | | | | |
|---|---|---|---|---|
| Alhydrogel ™ ‘85’ | 2.4 | 119 | 176 | 251 |
| Autoclaved | | | | |
| Rehydragel ™ HS | 3.4 | 144 | 211 | 306 |
| Alhydrogel ™ ‘85’ | 3.1 | 148 | 223 | 335 |
| Rehydragel ™ HS | 3.4 | 126 | 187 | 271 |
| Rehydragel ™ HS | 3.4 | 144 | 225 | 352 |
| Rehydragel ™ HS | 3.4 | 192 | 277 | 406 |

Tetanus TTS03-FA4

| | | | | |
|---|---|---|---|---|
| Rehydragel ™ HS | 3.4 | 169 | 249 | 371 |

Tetanus TTS03-FA3

| | | | | |
|---|---|---|---|---|
| Rehydragel ™ HS | 3.4 | 191 | 270 | 386 |

Tetanus TTS02-FA21

| | | | | |
|---|---|---|---|---|
| Rehydragel ™ HS | 3.4 | 178 | 263 | 394 |

### Example 3

### Clinical Study

Detailed Title

A phase I/II, double-blind, randomized, multicentre study to evaluate the safety and immunogenicity of new formulations of GlaxoSmithKline Biologicals' DTPa-HBV-IPV/Hib vaccine when administered to healthy infants as a primary vaccination course at 2, 3 and 4 months of age.

Indication

Primary immunization of healthy infants in the first year of life against diphtheria, tetanus, pertussis, hepatitis B, poliomyelitis and Haemophilus influenzae type b (Hib) diseases.

Rationale for the Study and Study Design

GSK Biologicals has validated two new formulations of DTPa-HBV-IPV/Hib (respectively containing D and T antigens adsorbed onto different aluminium salts of the invention) in pre-clinical setting and is now progressing them to clinical evaluation
Formulation A of the vaccine (D_{A}T_{A}Pa-HBV-IPV/Hib) contains diphtheria (D) and tetanus (T) antigens detoxified in presence of an amino acid and adsorbed on once-autoclaved and irradiated aluminium hydroxide adjuvant determined to have, on average, an adsorption capacity of 3.1 and a zeta potential of 16, while the other antigens are identical to those present in the licensed formulation of Infanrix hexa.
Formulation B of the vaccine (D_{B}T_{B}Pa-HBV-IPV/Hib) contains diphtheria (D) and tetanus (T) antigens detoxified in presence of an amino acid and adsorbed on irradiated aluminium hydroxide adjuvant determined to have, on average, an adsorption capacity of 3.6 and a zeta potential of 20, while the other antigens are identical to those present in the licensed formulation of Infanrix hexa.

The present trial is a phase I/II study aimed at evaluating the safety and immunogenicity of two new formulations when administered as a primary vaccination course to healthy infants at 2, 3 and 4 months of age.

The licensed formulation of Infanrix hexa vaccine has been used as a benchmark to establish non-inferiority of immune response to all vaccine antigens.

The purpose of the study is to demonstrate that the immunogenicity of at least one of these new formulations is non-inferior to the immunogenicity of the currently licensed formulation of the vaccine regarding diphtheria, tetanus, hepatitis B, polyribosyl-ribitol-phosphate (PRP) and pertussis antigens.

The study adopts a randomized double-blind design to forestall any chance of a bias in the evaluation of the new formulations.

Objectives

Primary

To demonstrate that the immunogenicity of at least one DTPa-HBV-IPV/Hib formulation is non-inferior to the licensed formulation in terms of seroprotection rates to diphtheria, tetanus, hepatitis B and PRP antigens and in terms of antibody geometric mean concentrations (GMCs) for pertussis antigens one month after the third dose of the primary vaccination.

Secondary
To assess the immunological response to the study vaccines in terms of seroprotection status, seropositivity status and antibody concentrations or titres, one month after the third dose of the primary vaccination.
To assess the immunological status towards diphtheria, tetanus pertussis and polio antigens in terms of seroprotection status, seropositivity status and antibody concentrations, before the first dose of the primary vaccination.
To assess the immunological response to pertussis antigens in terms of vaccine response, one month after the third dose of the primary vaccination.
To assess the safety and reactogenicity of the study vaccines in terms of solicited and unsolicited, local and general symptoms and serious adverse events.

Study Design
Experimental design: Phase I/II, double-blind, randomized, multicentre study with three parallel groups.
Duration of the study, per subject, was approximately three months.
Control: GSK Biologicals' DTPa-HBV-IPV/Hib vaccine (Infanrix hexa).
Vaccine schedule: All subjects received three doses of one of the three formulations of DTPa-HBVIPV/Hib at 2, 3 and 4 months of age, according to their group allocation.
In addition, all subjects received three doses of Wyeth-Lederle's 13-valent pneumococcal vaccine (Prevenar13) at 2, 3 and 4 months of age.

Treatment groups: Synopsis Table 1 presents the study groups and the vaccines administered in the study.

SYNOPSIS TABLE 1
Treatment groups

| | | Study Groups | | |
|---|---|---|---|---|
| Treatment | | Form A | Form B | Control |
| identifier | Vaccine name | Group | Group | Group |
| Formulation A | D_{A}T_{A}Pa-HBV-IPV | X | | |
| | Hib | | | |
| Formulation B | D_{B}T_{B}Pa-HBV-IPV | | X | |
| | Hib | | | |
| Infanrix hexa | DTPa-HBV-IPV | | | X |
| | Hib | | | |
| Prevenar 13 | Prevenar 13 | X | X | X |

Treatment allocation: Randomized 1:1:1.
Blinding: This is a double-blind study.
Blood sampling: A blood sample was taken from all subjects at the following two sampling time points:
   Before the first vaccine dose (Pre-Pri), a blood sample of at least 3.5 ml was collected;
   One month after the third vaccine dose (Post-Pri), a 5 ml blood sample was collected.
Type of study: Self-contained.
Data collection: Remote data entry (RDE) using electronic case report forms (eCRFs).

Number of Subjects
The target sample size was 720 subjects (240 in each group) to provide 648 subjects (216 in each group) evaluable for immunogenicity. The actual according-to-protocol (ATP) cohort for immunogenicity was 651 (Formulation A=215; Formulation B=217; Control=219).

Endpoints

Primary
Immunogenicity with respect to the components of the study vaccines.
   Anti-diphtheria, anti-tetanus, anti-HBs and anti-PRP seroprotection status one month after the third dose of primary vaccination.
   Anti-pertussis toxoid (anti-PT), anti-filamentous haemagglutinin (anti-FHA) and anti-pertactin (anti-PRN) antibody concentrations one month after the third dose of primary vaccination.

Secondary
Immunogenicity with respect to the components of the study vaccines.
   Anti-diphtheria, anti-tetanus, anti-HBs, anti-poliovirus type 1, anti-poliovirus type 2, anti-poliovirus type 3, anti-PRP, anti-PT, anti-FHA, anti-PRN, anti-pneumococcal serotypes 1, 3, 4, 5, 6A, 6B, 7F, 9V, 14, 18C, 19A, 19F, 23F, antibody concentrations or titres, and seroprotection and/or seropositivity status one month after the third dose of primary vaccination.
   Vaccine response to PT, FHA and PRN one month after the third dose of primary vaccination.
   Anti-diphtheria, anti-tetanus, anti-PT, anti-FHA, anti-PRN, anti-poliovirus type 1, anti-poliovirus type 2 and anti-poliovirus type 3 antibody concentrations or titres and seroprotection and/or seropositivity status before the first dose of primary vaccination.
Solicited local and general adverse events.
   Occurrence of solicited local symptoms during the 8-day (Day 0-7) follow-up period after each vaccination.
   Occurrence of solicited general symptoms during the 8-day (Day 0-7) follow-up period after each vaccination.
Unsolicited adverse events.
   Occurrence of unsolicited AEs during the 31-day (Day 0-30) follow-up period after each vaccination, according to the Medical Dictionary for Regulatory Activities (MedDRA) classification.
Serious adverse events.
   Occurrence of serious adverse events from Dose 1 up to study end.

Results
Previously a phase II trial (randomized, observer-blind, self-contained, primary immunization, multicentre study) was conducted of a reformulated DTPa-HBV-IPV/Hib vaccine containing D and T antigens produced by a new process and adsorbed onto the aluminium hydroxide Alhydrogel™π(re-autoclaved). 144 infants received the test vaccine and 149 the licensed control at 2, 3 and 4 months. One month after the third vaccine dose, D and T seroprotection was non-inferior to the licensed control. However, the non-inferiority criteria in terms of seroprotection rates for HBV and in terms of GMC ratio for anti-FHA and anti-PRN were not met.
In the present study, Formulation A appeared to represent a substantial improvement relative to the test formulation in the above-mentioned previous trial. In addition to excellent immunogenicity for D and T (non-inferiority criteria met for D and T seroprotection), the HBV seroprotection rate was significantly higher than in the previous trial and was non-inferior relative to the licensed formulation. The same trend to higher immune response in terms of GMCs was observed for D, T and HBV antigens as compared to the previous trial. The anti-FHA GMC similarly met non-inferiority criteria, whilst the anti-PRN GMC showed a modest improvement over the previous trial and marginally exceeded the non-inferiority cut-off. Formulation B also showed non-inferiority for seroprotection rates for D, T, HBV and for anti-FHA GMCs, but anti-PRN GMCs were lower than in Formulation A.

1. A process for producing an immunogenic composition comprising tetanus toxoid comprising the step of adsorbing the tetanus toxoid onto an aluminium salt particle wherein the aluminium salt particle has a protein adsorption capacity of between 2.5 and 3.7, 2.6 and 3.6, 2.7 and 3.5, or 2.8 and 3.4.

2. The process of claim 1 wherein the aluminium salt particle has a protein adsorption capacity of between 2.5 and 3.5, 2.6 and 3.4, 2.7 and 3.3 or 2.9 and 3.2.

3. The process according to claim 1 wherein the aluminium salt has a crystal size of between 2.8 and 5.7 nm as measured by X-ray diffration.

4. The process according to claim 3 wherein the aluminium salt has a crystal size of between 3.3 and 5.7 nm as measured by X-ray diffraction.

5. The process according to claim 1 wherein the aluminium salt particle has a protein adsorption capacity of between 2.5 and 3.6 and a crystal size of between 2.9 and 5.6 nm.

6. A process for producing an immunogenic composition comprising tetanus toxoid comprising the step of adsorbing the tetanus toxoid onto an aluminium salt particle wherein the aluminium salt particle has a crystal size of between 2.8 and 5.7 nm as measured by X-ray diffraction.

7. The process according to claim 1 for producing an immunogenic composition comprising tetanus toxoid further comprising the step of adsorbing the diphtheria toxoid onto a second aluminium salt particle wherein the second aluminium salt particle to which the diphtheria toxoid is adsorbed has a protein adsorption capacity of between 2.5 and 3.5, 2.6 and 3.4, 2.7 and 3.3 or 2.9 and 3.2.

8. The process according to claim 7 wherein the second aluminium salt particle to which the diphtheria toxoid is adsorbed has a crystal size of between 2.8 and 5.7nm as measured by X-ray diffraction.

9. The process according to claim 1 for producing an immunogenic composition comprising tetanus toxoid comprising the step of adsorbing a diphtheria toxoid onto a second aluminium salt particle wherein the second aluminium salt particle has a crystal size of between 2.8 and 5.7nm as measured by X-ray diffraction.

10. The process according to claim 2 wherein the aluminium salt particle has a Zeta potential at pH 7 of between about 17 and 23 mV, 18 and 22 mV or 19 and 21 mV.

11. The process according to claim 1 wherein the aluminium salt particle has a Zeta potential at pH 7 of between about 14 and 22 mV, 15 and 21 mV or 16 and 20 mV.

12. The process according to claim 7 wherein the diphtheria toxoid and tetanus toxoid are adsorbed separately onto the aluminium salt particle.

13. The process according to claim 1 wherein the tetanus toxoid has been produced by chemically detoxifying tetanus toxin.

14. The process according to claim 1 wherein the tetanus toxoid has been produced by genetically detoxifying tetanus toxin.

15. The process according to claim 7 wherein the diphtheria toxoid has been produced by chemically detoxifying diphtheria toxin.

16. The process according to claim 7 wherein the diphtheria toxoid has been produced by genetically detoxifying diphtheria toxin.

17. The process according to claim 1 further comprising the step of formulating the immunogenic composition with an inactivated polio vaccine.

18. The process according to claim 1 further comprising the step of formulating the immunogenic composition with pertactin.

19. The process according to claim 1 further comprising the step of formulating the immunogenic composition with filamentous haemagglutinin.

20. The process according to claim 1 further comprising the step of formulating the immunogenic composition with pertussis toxoid.

21. The process according to claim 1 further comprising the additional step of formulating the immunogenic composition with inactivated polio vaccine, pertactin, filamentous haemagglutinin or pertussis toxoid, adsborbed onto a second aluminium salt particle wherein the second aluminium salt particle has a Zeta potential at pH 7 of between about 17 and 23 mV, 18 and 22 mV or 19 and 21 mV.

22. The process according to claim 21 wherein the second aluminium salt particle has a protein adsorption capacity of between 2.5 and 3.5, 2.6 and 3.4, 2.7 and 3.3 or 2.9 and 3.2.

23. The process according to claim 22 wherein the second aluminium salt particle has a crystal size of between 2.8 and 5.7nm as measured by X-ray diffraction.

24. The process according to claim 1 wherein the aluminium salt is aluminium hydroxide.

25. The process according to claim 1 wherein the aluminium salt has not been autoclaved.

26. The process according to claim 1 wherein the aluminium salt has been autoclaved only once.

27. The process according to claim 26 wherein the aluminium salt is autoclaved for the minimum time necessary to achieve sterilisation.

28. The process according to claim 1 wherein the aluminium salt is sterilised by irradiation.

29. The process according to claim 1 further comprising the step of formulating the immunogenic composition with a capsular saccharide from Haemophilus influenzae type b.

30. The process according to claim 1 further comprising the step of formulating the immunogenic composition with a hepatitis B surface antigen.

31. The process according to claim 1 further comprising the step of formulating the immunogenic composition with a capsular saccharide from Haemophilus influenzae type b or with a hepatitis B surface antige, wherein the aluminium particle is aluminium phosphate.

32. The process according to claim 31, wherein the aluminium salt particle has a protein adsorption capacity of between 2.5 and 3.7, 2.5 and 3.5, 2.6 and 3.6, 2.6 and 3.4, 2.7 and 3.5, 2.7 and 3.3, 2.8 and 3.4 or 2.9 and 3.2 mg BSA/mg aluminium salt.

33. A process for making a vaccine comprising one or more of diphtheria toxoid, tetanus toxoid, pertussis toxoid, filamentous haemagglutinin and pertactin, wherein one or more of diphtheria toxoid, tetanus toxoid, pertussis toxoid, filamentous haemagglutinin and pertactin are adsorbed to an aluminium adjuvant, wherein said adjuvant is sterilised by a process which comprises a step of irradiation and comprises no steps of autoclaving.

34. A process according to claim 33 wherein tetanus toxoid and/or diphtheria toxoid are present and are adsorbed to said aluminium adjuvant.

35. A process according to claim 33 or 34 wherein diphtheria toxoid, tetanus toxoid, pertussis toxoid, filamentous haemagglutinin and pertactin are all present and are adsorbed to said aluminium adjuvant.

36. An immunogenic composition made by the process of claim 1.
